(19) United States
(12) Patent Application Publication
(10) Pub. No.: US 2009/0203092 A1
(43) Pub. Date: Aug. 13, 2009

(54) Liquid Biofuel Mixture and Method and Device for Producing The Same

(76) Inventors: Peter Eisner, Freising (DE); Andreas Stäbler, München (DE); Andreas Malberg, München (DE); Michael Menner, Eichenau (DE); Michael Frankl, München (DE)

Correspondence Address: RENNER KENNER GREIVE BOBAK TAYLOR & WEBER, FIRST NATIONAL TOWER FOURTH FLOOR, 106 S. MAIN STREET, AKRON, OH, 44308, US

(21) Appl. No.: 11/884,468

(22) PCT Filed: Nov. 30, 2005
(86) PCT No.: PCT/DE2005/002156

§ 371 Date: Mar. 10, 2009

(30) Foreign Application Priority Data
Feb. 17, 2005 (DE) 10 2005 007 369.7

(51) Int. Cl. C12P 7/64; C12M 1/00

(52) U.S. Cl. 435/134; 435/289.1; 435/297.1

(57) ABSTRACT

The present invention relates to a biofuel mixture comprising a fraction of fatty acid alkyl esters and at least one fraction of bound glycerol in an amount of ≧1 wt %, based on the glycerol backbone, and a method and a device for producing said biofuel mixture.

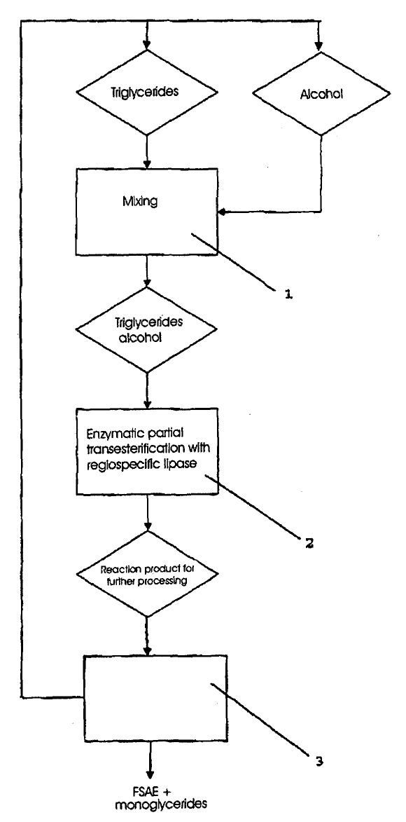

The biofuel mixture can be produced inexpensively, can also be utilized as a fuel in diesel engines without additional heating and can be added to conventional diesel fuel.

### TECHNICAL AREA OF USE

The present invention relates to a liquid biofuel mixture based on fatty acid alkyl esters and a method and a device for producing same. This fuel is suitable in particular as an additive for conventional fuels such as diesel or gasoline. Direct use of the fuel mixture as a fuel for internal combustion engines is also possible.

The term biofuels as used below is understood to refer to liquid fuels obtained from renewable raw materials. Examples of biofuels include animal fats, vegetable oils and liquids produced from vegetable or animal raw materials such as fatty acid alkyl esters from catalytic transesterification of fats and oils, bioethanol from fermentation of starch, sugar or celluloses or methanol from gasification of raw materials containing fat, starch, sugar or cellulose.

From an ecological standpoint, use of such renewable fuels is preferable to use of fossil fuels. For this reason, some of the so-called biofuels are already being added to traditional fuels such as diesel or gasoline today in order to improve the ecological balance of the fuels and also to comply with legal requirements.

### STATE OF THE ART

Biofuels and biofuel mixtures based on vegetable oil or animal fat are described, for example, in DE 4116905 C1, WO 95/25152 A1, EP 855436 A2 and U.S. Pat. No. 5,713,965 A. These publications disclose in particular mixtures of rapeseed [canola] oils with gasoline or diesel to which an additional substance is added. DE 4116905 C1 describes this additional component as being an alcohol; WO 95/25152 A1 describes an alkyl ester of a short-chain fatty acid with a maximum chain length of six carbons and EP 855436 A2 describes this as being an acetal.

The aforementioned documents also indicate that biological fats and oils cannot be used as fuels in the condition in which they are obtained industrially in their particular extraction process. Additives must be used and/or changes must be brought about in the physical and chemical properties, but high-priced additives, the cost of acquisition of which is significantly greater than the cost of conventional liquid fuels, in particular increase the cost of these biofuels and often make their use uneconomical.

WO 01/29154 A1 describes direct use of animal fat wastes in internal combustion engines as an economical approach. However, it is also known from the state of the art that direct use of renewable fats or oils in internal combustion engines leads to problems in the internal combustion process and results in deposits due to incomplete combustion because of the high viscosity and low cetane number.

At the present point in time, vegetable oil, animal fat, bioethanol and biodiesel are available as liquid biofuels.

Bioethanol is obtained by a fermentation process from raw materials present in plants. Carbohydrates are cleaved with the help of microorganisms and converted to ethanol by way of several intermediates. Since ethanol still contains at least 5% water in this process, it must be converted to an absolute form, usually with toluene, following the fermentation process.

The ethanol/toluene mixture is usually referred to as bioethanol and is a substitute for gasoline as a fuel. However, pure bioethanol cannot be used in traditional engines. A modification is necessary for combustion. However, it is possible to use a mixture, usually 95% gasoline and 5% bioethanol, with no problem.

Bioethanol has the advantages of a high octane rating, a high efficiency in combustion and low emissions.

The main disadvantage of bioethanol is the low energy density, the poor ecobalance, the low efficiency of the fermentation process and the use of the toluene as an aromatic agent. Furthermore, high carbon dioxide avoidance costs must also be taken into account in bioethanol production. For these reasons, use of bioethanol as a gasoline additive is disputed for both ecological and economic reasons.

Vegetable oils are a substitute for diesel fuel. They have the best ecobalance of all biofuels and have a comparatively high energy density of 38 MJ/kg (diesel 43 MJ/kg). Nevertheless, oils have not yet been successful as a fuel because their use in diesel engines has proven technically complicated. The most serious problem is the high viscosity of the substances. Because of this, there is an increase in the pump internal pressure and a change in the injection behavior. This may lead to damage to gaskets, in the combustion chamber, on the sparkplugs and the pistons. The high viscosity may also lead to incomplete combustion of the fuel, as does the poor ignition performance. Therefore, oil and/or fat as well as combustion residues remain in the combustion chamber and are deposited on the piston and nozzles. Furthermore, resinification occurs with prolonged operation using vegetable oils.

Another problem in using vegetable oils as a diesel substitute is the high corrosiveness of the free fatty acids. Free fatty acids are formed in the chemical and biological decomposition of the fat molecules, attacking mainly hoses and seals, but also attacking metal components in the fuel system after lengthy use.

For these reasons, combustion of vegetable oils and vegetable oil/diesel mixtures in commercial engines is impossible. Although these difficulties can be relieved by modification of the engine, this makes vegetable oil of little interest economically as a fuel.

Animal fats have the same disadvantages as vegetable oils. However, animal fats have a much higher viscosity and also form fatty acids that are released much more rapidly than is the case with vegetable oils, so their use as fuels is possible only in heavy oil burners with rotary atomizers.

The disadvantages discussed above can be largely overcome by chemical transesterification of vegetable oils with monovalent alcohols to form fatty acid alkyl esters (FAAEs, biodiesel). Biodiesel has an energy density similar to that of vegetable oil and can be used in almost all diesel engines of a new design thanks to its diesel-like viscosity and cetane number. Biodiesel is biodegradable and is not a hazardous substance due to its relatively high flash point.

Another advantage of FAAEs is their greatly improved emission values in comparison with fossil diesel. The sulfur dioxide, hydrocarbon and soot particulate emissions in particular are greatly reduced. Only the nitrogen oxide emission is slightly elevated.

The main disadvantage of biodiesel is its complex and expensive production process. Because of the numerous processing steps that are complicated in terms of both energy and process engineering for the two product biodiesel and glycerol, these have a strongly negative effect on the ecobalance and profitability of FAAE production, especially since only approximately 89% (% by weight) of the reaction products can be utilized as fuel. The 11% (percent by weight) glycerol that is formed as a second phase in biodiesel production must be separated and eliminated in a complex process. Because of the product processing, production in decentralized plants is not economically feasible. Therefore, at the present time, biodiesel is being produced almost exclusively in plants having a throughput of more than 10,000 tons per year. This causes a not insignificant logistics complexity.

Furthermore, the low winter stability and oxidation stability of FAAEs are another problem.

Biodiesel is produced by catalytic transesterification of vegetable oil. Dehydrated, deacidified and degummed oil with a molar alcohol excess (usually methanol) of 6:1 is reacted using 1 wt % catalyst (usually KOH) at a temperature above the boiling point of the alcohol. The fatty acids present in the fat molecule are split off catalytically and react with the alcohol that is present to form fatty acid alkyl esters. Fats and oils are triglycerides, i.e., one fat molecule contains three fatty acids bound to one glycerol molecule. Thus, in a complete transesterification reaction, such as that performed in the production of biodiesel, three molecules of biodiesel and one molecule of glycerol are formed per molecule of fat or oil.

Intermediate products of the reaction include mono- and diglycerides. Mono- and diglycerides consist of a basic glycerol structure, hereinafter also referred to as the glycerol backbone, linked to one fatty acid (monoglyceride) or two fatty acids (diglyceride). Since polar hydroxide groups as well as apolar hydrocarbon chains are present in mono- and diglycerides, they have amphiphilic properties and in organic solutions they almost always change the polarity of the solvent.

The transesterifi cation process requires a reaction time of approximately eight hours, yielding a conversion of approximately 98%.

Following the reaction, the glycerol which is formed and is insoluble in FAAE is removed from the biodiesel by means of a phase separator and is used as an industrial or pharmaceutical raw material after chemical and distillative purification.

The excess alcohol present in the FAAE is separated by distillation and recycled to the process. Then the biodiesel is washed with water to remove soaps that are formed as well as the catalyst and glycerol residues, and then is dried.

The object of the present invention is to make available a biofuel mixture and a method and a device for producing same, with which the aforementioned disadvantages of fuels according to the state of the art can be avoided, especially the high production costs. The biofuel mixture should have a lower viscosity than vegetable oil so that the fuel can also be utilized in diesel engines without additional heating and can be added to conventional diesel fuel. It should also be liquid and should form a single phase at low temperatures to achieve a high measure of stability in storage.

### DESCRIPTION OF THE INVENTION

This object is achieved with the biofuel mixture according to Patent claim 1, the methods according to patent claims 11 and 21 and the device according to patent claim 24. Advantageous compositions of the biofuel mixture as well as embodiments of the methods and the device for production of same are the subject of the subclaims or can be derived from the following description and the exemplary embodiments.

The inventive biofuel mixture contains at least one fraction of fatty acid alkyl esters and one fraction consisting of bound glycerol in the form of mono- and diglycerides and/or triglycerides. The amount of bound glycerol is at least 1 wt % in the fuel mixture, based on the glycerol backbone (empirical formula of the glycerol backbone: C₃H₅O₃; molecular weight 89 g/mol), preferably between 3 and 10 wt %. Higher concentrations, which may be desired under some circumstances, can be obtained by adding glycerides.

It has surprisingly been found that such biofuel mixtures containing the present amounts of monoglycerides and/or diglycerides are capable of more than doubling the solubility of free glycerol in FAAE. In conventional transesterification of fats and oils to alkyl esters, as mentioned above, glycerol separates out as a second phase from the biofuel. This phase must be separated from the alkyl esters at great expense. The glycerol, which is a natural constituent of oils and fats, can be utilized together with the other fractions in the combustion process in the inventive biofuel mixture. The yield due to the joint use of glycerol in the fuel (especially in the form of glycerides) is thus increased by approximately 10%, which brings definite cost advantages.

The inventive biofuel mixture is also capable of keeping more than 40 wt % fats or oils in solution and thus permitting joint use of these substances in the fuel mixture without forming additional phases or having to separate additional phases.

The biofuel mixture also has lower exhaust gas values with regard to hydrocarbons, carbon monoxide and soot particles in comparison with biodiesel.

It has been found that monovalent alcohols such as methanol or ethanol can also be dissolved very well in the inventive biofuel mixture. Thus, the alcohol, which is not completely consumed in the synthesis process of the fatty acid alkyl ester, can be left in the biofuel mixture or a monovalent alcohol may be added to the mixture. This leads to a decline in the viscosity and to an improvement in the cold stability.

In an advantageous embodiment of the method, bioethanol is used as the alcohol for the transesterification.

It is also found that the miscible of the biofuel mixture with mineral fuels is improved by the mono- and diglycerides contained therein in comparison with traditional biodiesel.

The biofuel mixture can be mixed with mineral fuel or traditional biodiesel in any ratio, diluted in the process and used as a fuel. It is thus possible to adjust a lower concentration of bound glycerol in the fuel finally used. It is also possible to achieve dilution of the inventive fuel mixture by adding additives from diesel fuel or biodiesel already before the transesterification of the vegetable oil.

To improve the oxidation stability and behavior at low temperatures, it is possible to add state-of-the-art fuel additives to the fuel according to this invention.

It is also recommended that mono- and diglycerides, which are formed in the transesterification of vegetable oil to fatty acid alkyl esters, for example, should be added to the biofuel mixture. However, it is also possible and may be advantageous under some circumstances to use mono-, di- and triglycerides which originate from another source or are of synthetic origin. Thus, mono- and diglycerides, which contain fatty acids with fewer than 10 carbon atoms in the fatty acid molecule, may also be used in the biofuel mixture. This may offer particular advantages in reducing the viscosity.

Two methods are given below for production of the proposed biofuel mixture.

One possible production process is based on a partial transesterification of triglycerides.

To do so, purified and optionally dehydrated fat or oil is mixed with a monoyalent alcohol and reacted by adding a suitable catalyst. In doing so, the fat, oil, alcohol and catalyst may of course also consist of mixtures of different substances.

The ratio of FAAE, mono-, di- and optionally triglycerides in the reaction product may be adjusted through the dwell time, the catalyst and the amount of alcohol used.

One or more regiospecific lipases are preferably used as catalyst. It is advantageous to use sn-1,3-regiospecific lipases as the catalyst. Such lipases preferably split off the first and third fatty acids from the triglyceride. This forms a mixture of mono- and diglycerides together with FAAEs in the presence of alcohols.

For adjusting the desired fuel properties, e.g., the viscosity, however, it is also possible to add an unspecified catalyst, in which then the required amount of mono- and/or diglycerides in the reaction product can be achieved, for example, by premature termination of the reaction or by adding a substoichiometric amount of alcohol. The resulting glycerol remains in solution due to the mono- and diglycerides but, if necessary, it may also be separated from the fuel with suitable separation methods. The FAAE is formed in parallel with this reaction. This constituent of the reaction product reduces the viscosity of the biofuel mixture.

In addition, it has been found that the alcohol consumption is decreased by 33-50% in comparison with traditional biodiesel production because the alcohol glycerol remains in the biofuel mixture and need not be replaced.

The catalyst and/or the catalyst mixture may be in free form or in a supported catalyst system. Supported catalysts have the advantage that they can be used over several reaction cycles. Because of the comparatively high price, this is advantageous especially when using lipases as the catalyst.

The device proposed for production of the biofuel therefore has, in addition to a mixing apparatus for mixing triglycerides with alcohol, a reactor to hold the mixture, containing one or more supports with one or more immobilized regiospecific lipases. This may be, for example, a stirred reactor or a fixed bed reactor.

In one embodiment, a separation device is connected downstream from the reactor for separating a fraction containing bound glycerol and/or alcohol from the product obtained by the reaction. This fraction which is separated is preferably recycled back to the mixing apparatus so that no waste products are formed in the production process. It is also possible to send the separated fraction for separate utilization. The separation apparatus may be, for example, a distillative separation apparatus or a membrane separation apparatus or a crystallization-separation apparatus or an adsorption-separation apparatus or an extraction-separation apparatus.

The process temperature for production of the biofuel mixture depends on the catalyst used and the triglyceride used. However, it usually varies between 20° C. and 120° C.

The reaction rate depends on the catalyst concentration and the catalyst used. The reaction time and/or dwell time is selected as a function of the desired fuel properties.

To increase the fatty acid alkyl ester yield, it is advantageous during the reaction to remove the water which is in the system as well as the water formed during the transesterification process by state-of-the-art methods. State-of-the-art methods include, for example, drying by means of a molecular sieve or sodium sulfate or removal of water by pervaporation. Removal of water during the transesterification process also offers the advantage that the formation of free fatty acids is reduced.

A downstream purification of the fuel is not necessary except for removal of the free and/or supported catalyst. However, purification may be performed to adjust certain properties, e.g., to increase the viscosity by removing the residual alcohol. In addition, it may be advantageous to remove part of the bound glycerol from the fuel mixture to adjust a lower viscosity. This may be accomplished with the help of the state-of-the-art methods, e.g., by membrane methods, crystallization, adsorption or extraction, e.g., with water or other polar or amphiphilic liquids.

It is also possible to subject some of the separated di- or triglycerides to a nonspecific transesterification after the regiospecific lipase treatment. This makes it possible to obtain a larger amount of monoglycerides under some circumstances.

In addition to production of the biofuel mixture by partial transesterification, the biofuel mixture may also be obtained by adding mono- and diglycerides, optionally also alcohols and triglycerides, to pure, i.e., commercial FAAEs. The amounts of glycerides and alcohols used depend on the desired properties. For the most advantageous possible fuel properties, i.e., a low viscosity and a high cetane number, a high FAAE content of >50 wt % is advantageous, especially preferably >60 wt %, and in some cases even >80 wt %. If use as a solvent is also intended, then a high FAAE content, preferably 22 50 wt %, and a high monoglyceride content, preferably >25 wt % should be the goal. The amount of residual fat for this application should be as low as possible, preferably <2 wt %.

It is advantageous if both mono- and diglycerides are present in the fuel. If only monoglycerides are present, for example, the monoglycerides may crystallize out. Adding di- and/or triglycerides inhibits crystallization and thus ensures a good stability in storage.

### METHODS OF IMPLEMENTING THE INVENTION

The fuel is illustrated below on the basis of two examples of alkyl esters.

### EXAMPLE 1

To 100 g fatty acid methyl ester (biodiesel) is added 50 g of a mixture of monoglycerides (45 wt %), diglycerides (20 wt %) and triglycerides (35 wt %). This glyceride mixture can be obtained commercially. The biofuel mixture can be used as a fuel.

### EXAMPLE 2

To 100 g vegetable oil are added 3.5 g methanol (other monovalent or divalent alcohols are also possible) and 1 g of a sn-1,3-regiospecific lipase. The mixture is mixed for nine hours at the temperature of the highest lipase activity. After nine hours, 3.5 g methanol is added again. The system is stirred for fifteen hours more at the above optimal lipase temperature, resulting in a clear solution of monoglycerides, diglycerides, FAAEs and vegetable oil containing a few wt % methanol dissolved in it.

The figure shows in highly schematic form the components of an exemplary apparatus for production of the biofuel mixture and the interaction of these components in the production process. First, triglycerides and alcohol are placed in a mixing apparatus 1 and combined there. The mixture of triglycerides and alcohol is then transferred to a stirred reactor or a fixed bed reactor 2. This may be accomplished via a connecting line between the mixing apparatus and the reactor 2. The mixture is brought in contact with sn-1,3-regiospecific lipases as the catalyst in reactor 2 to achieve a partial transesterification. The regiospecific lipases are present in immobilized form on one or more supports in the reactor. A mixture of fatty acid alkyl esters and monoglycerides, optionally also containing diglycerides and triglycerides, is obtained as the product of the reaction.

A residue of alcohol and triglycerides can be removed from the reaction product by distillation or by means of membrane separation techniques in a separation apparatus 3, optionally connected downstream from the reactor 2, and then recycled back to the process in the mixing apparatus 1.

1. A method for producing a biofuel Mixture comprising a fraction of fatty acid alkyl esters and at least one fraction of bound glycerol in an amount of ≧1 wt %, based on the glycerol backbone, by partial transesterification of triglycerides with the addition of alcohol at a temperature between 20° C. and 120° C., in which purified fat or oil is mixed with a monovalent and/or divalent alcohol and brought to reaction by adding a catalyst that is suitable for splitting off fatty acids and is bound to a support until obtaining a mixture of fatty acid alkyl esters and at least monoglycerides and/or diglycerides as the reaction products, said mixture being used directly as a fuel or as an additive for conventional fuels without separation of glycerol after removing the supported catalyst.

2. The method according to claim 1,
characterized in that
the partial transesterification is performed until obtaining a mixture of fatty acid alkyl ester, monoglycerides and/or diglycerides and at least triglycerides in a certain ratio as the reaction product.

3. The method according to claim 1 or 2,
characterized in that
the ratio of the reaction products is adjusted while the fat or oil is brought in contact with the monovalent and/or divalent alcohol and the catalyst.

4. The method according to claim 1 or 2,
characterized in that
the ratio of the reaction products is adjusted via the amount of monovalent and/or divalent alcohol with which the fat or oil is combined.

5. The method according to claim 1 or 2,
characterized in that
the ratio of the reaction products is adjusted through the type and/or amount of the catalyst.

6. The method according to claims 1 or 2,
characterized in that
one or more regiospecific lipases is used as the catalyst.

7. The method according to claims 1 or 2,
characterized in that
one or more nonspecific catalysts and/or nonspecific enzymes is used.

8. The method according to claims 1 or 2,
characterized in that
before and/or during the partial transesterification, dehydration is performed.

9. The device for performing the method according to claim 1,
comprising a mixing apparatus for mixing triglycerides with alcohol and a reactor for holding the mixture, containing one or more supports with one or more immobilized regiospecific lipases, whereby a separation apparatus for separating a fraction containing fat and/or oil and/or alcohol is connected downstream from the reactor and has at least one outlet for the fraction containing the fat and/or oil and/or alcohol, said outlet being connected to the inlet of the mixing apparatus.

10. The device according to claim 9,
characterized in that
the separation apparatus is a distillative separation apparatus or a membrane separation apparatus or a crystallization separation apparatus or an absorption separation apparatus or an extraction separation apparatus.

11. The device according to claim 9,
characterized in that
the reactor is a stirred reactor or a fixed bed reactor.

12-28. (canceled)
